(19) United States
(12) Patent Application Publication
(10) Pub. No.: US 2006/0047253 A1
(43) Pub. Date: Mar. 2, 2006

(54) Needle assembly

(76) Inventors: Michael H. Hayman, Metairie, LA (US); Kermit L. Roux JR., Metairie, LA (US)

Correspondence Address: KEATY PROFESSIONAL LAW CORPORATION;THOMAS S. KEATY, 2533 AMERICAN WAY, PORT ALLEN, LA, 70767, US

(21) Appl. No.: 10/924,628

(22) Filed: Aug. 24, 2004

(51) Int. Cl. A61M 5/32

(52) U.S. Cl. 604/272

(57) ABSTRACT

A needle assembly particularly suitable for prostate brachytherapy anesthesia and biopsy anesthesia procedure. The needle assembly has a cannula with a tapered distal end and a hub attached to a proximal end of the cannula. The cannula is provided with a plurality of spaced-apart openings extending through a wall of the cannula between the proximal end and the distal end for creating a pre-determined pattern of distribution in variable cylindrical patterns along the needle tract.

### BACKGROUND OF THE INVENTION

This invention relates to medical devices and more particularly to a needle assembly suitable for use in prostate brachytherapy anesthesia and provides the anesthesia.

The biopsy procedure is extensively used in modern medicine for detecting cell abnormalities, such as presence of cancerous growth. Patients with suspected carcinoma of the prostate are required to undergo a biopsy procedure wherein a number of samples of the tissue are taken from the area surrounding the prostate gland. In one study, 85 percent of the patients with carcinoma of the prostate were diagnosed using six sample techniques, while 97 percent of the patients were diagnosed using a ten-sample biopsy technique. As the trend to perform more biopsies progresses, it has become necessary to decrease the discomfort felt by the patient during the procedure. Although patient's perceptions of pain during prostate biopsy is variable; it is unusual to find a patient who reports the procedure as painless.

The prostate is a male accessory reproductive organ located in the pelvis. Immediately superior to the prostate, is the urinary bladder and directly inferior to the prostate is the penile urethra in the base of the penis. The urogenital diaphragm separates the prostate from the penile urethra. The prostatic urethra traverses the center of the gland and connects the urinary bladder to the penile urethra. Posterior to the prostate is the rectum. By inserting a finger into he rectum, a physician can palpate the prostate through the anterior wall of the rectum.

The architecture of the prostate gland is defined by three histologically separate zones: 1) the peripheral zone, 2) the central zone, and 3) the transitional zone. The peripheral zone is located posterially and laterally of the prostate gland and is most commonly biopsied with a technique known as the “transrectal ultrasound guided biopsy”. In this procedure an ultrasound probe is inserted into the rectum and biopsies are taken through the rectal wall using a special 18-gauge needle gun to insert the needle and take the biopsy. This procedure is recognized as a superior procedure.

The prostate is enervated by the neuromuscular bundles located on the posterior lateral portion of the prostate. These neurovascular bundles are derived from the inferior branches of the prostatic plexus, which are an offshoot of the inferior hypogastric plexus. In males, sympathetic and parasympathetic fibers pass through the structures of the perineum from the prostatic plexus. These fibers are derived from the upper three lumber segments and the pelvic sphlanic nerves, sacral segments 2-4 respectively. Terminal fibers communicate with the pudendal nerves.

With the presence of major nerve bundles in the area of investigation, the prostate gland becomes an extremely sensitive location. During biopsy, measures are taken to reduce discomfort of the patient. For instance, physicians use a prostatic block technique, wherein injections are given at three positions on each side of the prostate using a 20-gauge needle. Under ultrasound guidance, the needle is initially positioned at the base of the prostate, near the junction of the prostate and the seminal vesicle. The second injection site is at the mid portion of the prostate and the third injection site is at the apex of the prostate. This procedure is said to improve the pain score in patients and make the procedure more acceptable.

Another method is an intra-rectal Lidocaine administration for pain control during transrectal prostate biopsy. This method was reported as an improvement of the pain score as well.

However, there remains a need to reduce the number of injections during preparation of the site for a biopsy test to thereby reduce pain and discomfort of the patient during the complex procedure. The present invention contemplates provision of a device that can be used for this type of biopsy anesthesia.

### SUMMARY OF THE INVENTION

It is, therefore, an object the present invention to provide a needle assembly suitable for use in biopsy and anesthesia in the prostate region.

It is another object of the present invention to provide a needle assembly that can deliver local anesthesia through multiple locations along the length of the needle and thereby reduce the number of injections needed for numbing the area of investigation.

It is a further object of the present invention to provide a biopsy and anesthesia needle assembly that would allow to conduct the biopsy or anesthesia procedure in a more efficient manner, thereby reducing pain and discomfort of the patients.

These and other objects of the present invention are achieved through a provision of a needle assembly that has a hub and a needle cannula secured to the hub. The cannula has a lumen of a predetermined diameter and a tapered distal end. A plurality of spaced-apart openings is formed through the wall of the cannula between the proximal end, which is connected to the hub, and the tapered end. The through openings may be formed at about 90 degrees in relation to adjacent openings or may be formed in a linear pattern. When the liquid passes through the cannula it is distributed in a pre-determined pattern for delivery of anesthesia, medication or for performing a biopsy procedure.

The openings start at about 10 mm from a line of attachment of the hub to the cannula and continue with intervals of about 10 mm or more towards the distal end of the cannula. In the preferred embodiment, there are six such openings, although the number of openings may differ depending on the length of the needle.

### BRIEF DESCRIPTION OF THE DRAWINGS

Reference will now be made to the drawings, wherein like parts are designated by like numerals, and wherein

FIG. 1 is a perspective view of the needle assembly in accordance with the present invention.

FIG. 2 is a side plan view of the needle assembly of the present invention.

FIG. 3 is a side plan view of the needle assembly of the present invention, with the needle assembly turned 90 degrees as compared to the view of FIG. 2.

FIG. 4 is a detail view of the needle cannula showing an opening through the wall of the needle.

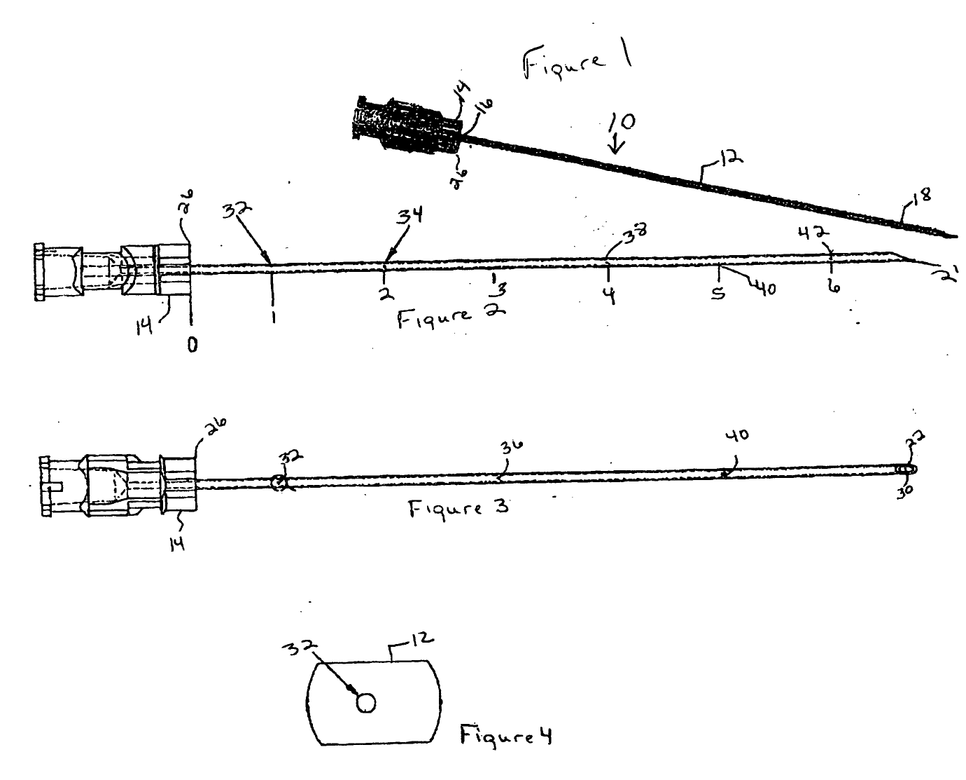

### DETAILED DESCRIPTION OF THE PREFERRED EMBODIMENT

Turning now to the drawings in more detail, numeral 10 designates the needle assembly in accordance with the present invention. The assembly 10 comprises a cannula portion 12 and a hub portion 14 connected to the needle portion by luer connection or other attachment means. The cannula portion 12 has an open proximal end 16, a distal open end 18 and a tip portion 20 extending from the distal end 18. The tip 20 is provided with a sharpened end 24. The hub 14 has a distal end 26, which is joined to the proximal end of the cannula 12 so that the lumen opening extending through the length of the cannula 12 is fluid communication with the hub 14.

The cannula 12 has an opening 30 in the distal end thereof, as shown in FIG. 3. The wall of the cannula 12 is perforated at different locations along the length of the needle cannula. For the purpose of illustration, numeral “0” designates the base line of connection between the proximal end 26 of the hub 14 and the cannula proximal end 16.

A first opening 32 is formed at about 10 mm from the base location 0. The second opening 34 may be formed through the wall of the cannula 12 at 90 degrees location around the cannula wall circumference in relation to the first opening 32. The second opening is designated by numeral 2 in FIG. 2; it is formed at about 23.74 mm from the base location “0”. The third opening 36 is formed through the cannula wall at a distance from the second opening 34 at a location designated by numeral 3 in FIG. 2. The third opening is formed at 90 degrees around the cannula circumference in relation to the second opening 34 and is located about 37.48 mm from the base location “0”. The fourth opening 38 is formed at location designated by numeral 4 in FIG. 2. The fourth opening 38 is removed by 51.22 mm from the base location “0” may be spaced by 90 degrees from the third opening 36.

The fifth opening 40 is found in a location designated by numeral 5 in FIG. 2. The opening 40 is formed farther along the cannula 12 and is made at a 90-degree angle to the opening 38. The location 5 is removed from the base location by about 64.96 mm.

The final sixth opening 42 is made at a location designated by numeral 6 in FIG. 2. The opening 42 is removed from the base location by 78.70 mm. There is a distance between the sixth opening 32 and the tip 24 of the cannula 12. In the preferred embodiment, the tip 24 is located at about 88.7 mm from the base location “0”. The openings 32, 34, 36, 38, 40 and 42 may be formed in a linear fashion along the cannula 12, depending on the preferred pattern of the liquid distribution.

The needle 10 is 14 gauge or less, although in the preferred embodiment uses 22 or 23 gauge. In use, the needle is inserted through the perineum or the rectum using a standard ultrasound work channel and placed in the periprostatic area near the neurovascular plexus. The tip 20 of the needle 10 is positioned at the base of the prostate near the junction of the prostate with the seminal vesicle. The openings 32, 34, 36, 38, 40, and 42 arranged in a spiral or linear fashion will allow delivery of local anesthesia infused through the needle to create a variable cylindrical distribution of anesthetic near the neurovascular bundle. This delivery of anesthetics, the total nerve block of the prostate and of the anterior rectal wall is easily achieved.

The open end 30 allows added introduction of medication from the needle into the tissue, assisted by the delivery of the anesthetic through the perforations at the plurality of sites distal from the open end 30. Similarly, other therapeutic agents will be delivered to a local site along the needle tract. An infusion of the therapeutic substance creates a pre-determined pattern of distribution along the needle tract. The needle has sufficient rigidity with the obturator in place to allow insertion through the skin of the perineum or the rectal wall.

Many changes and modifications can be made in the design of the present invention without departing from the spirit thereof. I therefore pray that my rights to the present invention be limited only by the appended claims.

I claim:

1. A needle assembly, comprising:
a hub having an open proximal end and a distal end; and
an elongated cannula having a proximal end, a distal end with a tapered tip and a lumen therethrough, said proximal end of the cannula being secured to the distal end of the hub for creating fluid communication between the cannula lumen and the hub open end, said cannula having at least one opening formed through a wall of the cannula and extending at an angle to the cannula lumen between the tapered tip and the proximal end.

2. The needle assembly of claim 1, wherein said cannula is provided with a plurality of through openings formed in the cannula wall, said openings being formed at about 90 degree angle in relation to immediately adjacent openings.

3. The needle assembly of claim 1, wherein said cannula is provided with six openings formed through the cannula wall, a first opening being formed at about 10 mm from a line of connection between the distal end of the hub and the proximal end of the cannula.

4. The needle assembly of claim 3, wherein a second opening being formed at about 23.74 mm from a line of connection between the distal end of the hub and the proximal end of the cannula.

5. The needle assembly of claim 3, wherein a third opening being formed at about 37.48 mm from a line of connection between the distal end of the hub and the proximal end of the cannula.

6. The needle assembly of claim 3, wherein a fourth opening being formed at about 51.22 mm from a line of connection between the distal end of the hub and the proximal end of the cannula.

7. The needle assembly of claim 3, wherein a fifth opening being formed at about 64.96 mm from a line of connection between the distal end of the hub and the proximal end of the cannula.

8. The needle assembly of claim 3, wherein a sixth opening being formed at about 78.70 nail from a line of connection between the distal end of the hub and the proximal end of the cannula.

9. A needle assembly, comprising:
a hub having an open proximal end and a distal end; and
an elongated cannula having a proximal end, a distal end with a tapered tip and a lumen therethrough, said proximal end of the cannula being secured to the distal end of the hub for creating fluid communication between the cannula lumen and the hub open end, said cannula being provided with a plurality of spaced-apart perforations formed in a wall of the cannula between the tapered tip and the proximal end for creating a pre-determined pattern of distribution of liquids passing through the cannula lumen.

10. The needle assembly of claim 9, wherein said cannula is provided with six perforations, each perforation extending at about 90 degree angle in relation to adjacent perforations.

11. The needle assembly of claim 9, wherein said cannula is provided with six perforations, a first of said perforation being formed at a distance of about 10 mm from a line of connection between the hub and the cannula, a second of said perforation being formed at a distance of about 23.74 mm from the line of connection between the hub and the cannula, a third perforation being formed at a distance of about 37.48 mm from the line of connection between the hub and the cannula, a fourth perforation being formed at a distance of about 51.22 mm from the line of connection between the hub and the cannula, a fifth perforation being formed at a distance of about 64.96 mm from the line of connection between the hub and the cannula, and a sixth perforation being formed at a distance of about 78.70 mm from the line of connection between the hub and the cannula.

12. A needle assembly, comprising:
a hub having an open proximal end and a distal end; and
an elongated cannula having a proximal end, a distal end with a tapered tip and a lumen therethrough, said proximal end of the cannula being secured to the distal end of the hub for creating fluid communication between the cannula lumen and the hub open end, said cannula being provided with six spaced-apart openings formed in a wall of the cannula between the tapered tip and the proximal end for creating a pre-determined pattern of distribution of liquids passing through the cannula lumen, a first of said openings being formed at about 10 mm distance from a line of connection between the hub and the cannula and other openings being formed with a distance of at least 10 mm between adjacent openings.

13. The needle assembly of claim 12, wherein said openings form a spiral pattern of distribution of liquids passing through the cannula.

14. The needle assembly of claim 12, wherein said opening form a linear pattern of distribution of liquids passing through the cannula.
